(19) United States
(12) Patent Application Publication
(10) Pub. No.: US 2008/0035166 A1
(43) Pub. Date: Feb. 14, 2008

(54) Aerosol device containing a hair composition

(75) Inventors: Christine Dupuis, Paris (FR); Jonathan Gawtrey, Boulogne (FR)

Correspondence Address: OBLON, SPIVAK, MCCLELLAND MAIER & NEUSTADT, P.C., 1940 DUKE STREET, ALEXANDRIA, VA, 22314, US

(73) Assignee: L'OREAL, Paris (FR)

(21) Appl. No.: 11/797,006

(22) Filed: Apr. 30, 2007

Related U.S. Application Data
(63) Continuation of application No. 10/748,181, filed on Dec. 31, 2003, now abandoned.
(60) Provisional application No. 60/448,110, filed on Feb. 20, 2003.

(30) Foreign Application Priority Data
Jan. 2, 2003 (FR) 0300003

(51) Int. Cl. A61K 8/91; A45D 34/04; A61Q 5/06; A61Q 5/12; B65D 83/14

(52) U.S. Cl. 132/221; 222/402.1; 424/47

(57) ABSTRACT

The invention relates to an aerosol device containing a hair composition for hair styling which comprises, in a cosmetically acceptable aqueous medium, at least one copolymer based on N-vinylpyrrolidone and N-vinylimidazole units having a molecular weight greater than 100 KDaltons, and dimethyl ether as propelling agent. The invention also relates to the use of this device and to a method of hair treatment.

### REFERENCE TO PRIOR APPLICATIONS

This application claims priority to U.S. provisional application 60/448,110 filed Feb. 20, 2003, and to French patent application 0300003 filed Jan. 2, 2003, both incorporated herein by reference.

### FIELD OF THE INVENTION

The present invention relates to an aerosol device comprising a hair composition for hair styling in an aqueous medium propelled by dimethyl ether, based on an N-vinylpyrrolidone and N-vinylimidazole copolymer, the use of the said device, and a method of treatment using the said device.

Additional advantages and other features of the present invention will be set forth in part in the description that follows and in part will become apparent to those having ordinary skill in the art upon examination of the following or may be learned from the practice of the present invention. The advantages of the present invention may be realized and obtained as particularly pointed out in the appended claims. As will be realized, the present invention is capable of other and different embodiments, and its several details are capable of modifications in various obvious respects, all without departing from the present invention. The description is to be regarded as illustrative in nature, and not as restrictive.

### BACKGROUND OF THE INVENTION

Hair compositions for shaping and/or holding hairstyles which are most widespread on the market for cosmetic products are spray compositions essentially consisting of a solution, most often an alcoholic solution, and one or more materials, generally polymer resins, whose function is to form bonding between the hair, called fixing materials, as a mixture with various cosmetic adjuvants. This solution is generally packaged either in an appropriate aerosol container pressurized with the aid of the propelling agent, or in a pump dispenser.

Numerous aerosol systems are known which are intended for fixing the hair, these systems containing, on the one hand, a liquid phase (or juice) and, on the other hand, a propelling agent. The role of the latter is to provide a pressure allowing spraying of the liquid phase, and its application to the hair in the form of a mist of dispersed droplets. With the aim of reducing the quantity of volatile organic compounds (VOC) such as alcohols and fluorinated hydrocarbons or Freons present in this type of composition to be sprayed and in order to be environmentally friendly, aqueous formulations propelled by dimethyl ether are more particularly of interest.

The liquid phase contains the fixing materials and an appropriate solvent. It is only after application to the hair that the liquid phase dries, allowing the formation of the bonding necessary for the fixing of the hair by the fixing materials. The bonding should be sufficiently rigid to ensure that the hair is held. However, it should also be sufficiently fragile so that the user, on combing or brushing the hair, destroys it without striking the scalp or damaging the hair.

Aerosol devices containing compositions based on dimethyl ether and on polymer containing N-vinyllactam units are already known and are described for example in Patent Application FR 2 771 925.

However, there is a real need to improve the fixing and the holding of the hair, that is to say to obtain bonding of better quality, while retaining good cosmetic properties, such as softness or disentanglement.

### SUMMARY OF THE INVENTION

The inventors have now discovered that the use of a copolymer based on N-vinylpyrrolidone and N-vinylimidazole units in aqueous hair compositions allows an improvement in the fixing of the hair while maintaining good cosmetic properties.

One subject of the present invention is therefore an aerosol device containing a fixing aqueous hair composition based on at least one copolymer based on N-vinylpyrrolidone and N-vinylimidazole units, and a propelling agent containing dimethyl ether.

Another subject of the invention is the use of the said device for applying a lacquer to the hair.

Another subject of the invention is a two-component product containing the abovementioned compositions.

Another subject of the invention is a method of hair treatment using the said device.

Other subjects, characteristics, features and advantages of the invention will appear more clearly on reading the description and the various examples which follow.

### DETAILED DESCRIPTION OF THE PREFERRED EMBODIMENTS

The present invention relates to an aerosol device containing a hair composition for hair styling which comprises, in a cosmetically acceptable aqueous medium, at least one copolymer based on N-vinylpyrrolidone and N-vinylimidazole units having a molecular weight (MW) greater than 100 KDaltons, and dimethyl ether as propelling agent.

The molecular weight of the copolymers of the invention is preferably between 100 and 5000 KDaltons, and still more preferably between 500 and 1500 KDaltons.

Preferably, the copolymer based on N-vinylpyrrolidone and N-vinylimidazole units has a Young's modulus greater than 600 MPa, even better between 600 and 10 000 MPa. This Young's modulus or modulus of elasticity is a constant defined by the ratio between the stress and the strain for a given material. It is measured at 25° C. on a film having a thickness of 500 μm and a relative humidity level of 48% by the LYOD L151 apparatus.

The preferred copolymers consist of 10 to 90% by weight of N-vinylpyrrolidone units and of 10 to 90% of N-vinylimidazole units, and still more preferably of 40 to 60% by weight of N-vinylpyrrolidone units and of 40 to 60% of N-vinylimidazole units.

Preferably, the copolymer used is that marketed under the name LUVITEC VPI 55 K61.

The hair composition for hair styling according to the invention preferably contains from 0.1 to 15% by weight of copolymer relative to the total weight of the composition.

According to the invention, the composition preferably contains, in addition, at least one fixing polymer other than the fixing copolymers of the invention. These fixing polymers make it possible to give a shape to the hair or to hold it.

The fixing polymers suitable for the invention are those generally used in the art. They are chosen in particular from anionic, cationic, amphoteric, and nonionic polymers, and mixtures thereof.

The fixing polymers may be soluble in a cosmetically acceptable medium or may be insoluble in this same medium and may be used in this case in the form of dispersions of solid or liquid particles of polymer (latex or pseudolatex).

The anionic fixing polymers generally used are polymers containing groups derived from carboxylic, sulphonic or phosphoric acid and have a number-average molecular mass of between about 500 and 5 000 000.

The carboxylic groups are provided by unsaturated mono- or dicarboxylic acid monomers such as those corresponding to the formula: 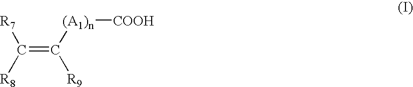
in which n is an integer from 0 to 10, A₁ denotes a methylene group, optionally linked to the carbon atom of the unsaturated group or to the neighbouring methylene group, when n is greater than 1, through a heteroatom such as oxygen or sulphur, R₇ denotes a hydrogen atom, or a phenyl or benzyl group, R₈ denotes a hydrogen atom, or a carboxyl or lower alkyl group, R₉ denotes a hydrogen atom or a lower alkyl group, a group —CH₂—COOH, or a phenyl or benzyl group.

In the abovementioned formula, a lower alkyl group denotes preferably a group having 1 to 4 carbon atoms, and in particular methyl and ethyl.

The anionic fixing polymers with carboxylic groups preferred according to the invention are:

A) the homo- or copolymers of acrylic or methacrylic acid or their salts and in particular the products sold under the names VERSICOL® E or K by the company ALLIED COLLOID and ULTRAHOLD® by the company BASF, the copolymers of acrylic acid and of acrylamide, the sodium salts of the polyhydroxycarboxylic acids;

B) the copolymers of acrylic or methacrylic acid with a monoethylene monomer such as ethylene, styrene, vinyl esters, acrylic or methacrylic acid esters, optionally grafted onto a polyalkylene glycol such as polyethylene glycol and optionally crosslinked. Such polymers are described in particular in French Patent 1 222 944 and German Application 2 330 956, copolymers of this type containing in their chain an acrylamide unit optionally N-alkylated and/or hydroxyalkylated as described especially in Luxembourg Patent Applications 75370 and 75371. There may also be mentioned the copolymers of acrylic acid and C₁-C₄ alkyl methacrylate and the terpolymers of vinylpyrrolidone, acrylic acid and C₁-C₂₀ alkyl, for example lauryl, methacrylate such as that sold by the company ISP under the name ACRYLIDONE® LM and the methacrylic acid/ethyl acrylate/tert-butyl acrylate terpolymers such as the product sold under the name LUVIMER® 100 P by the company BASF.

C) the copolymers derived from crotonic acid such as those containing in their chain vinyl propionate or acetate units and optionally other monomers such as methallyl or allyl esters, vinyl ether or vinyl ester of a linear or branched saturated carboxylic acid with a long hydrocarbon chain such as those containing at least 5 carbon atoms, it being possible for these polymers to be optionally grafted and crosslinked or alternatively another monomer which is a vinyl, allyl or methallyl ester of an α- or β-cyclic carboxylic acid. Such polymers are described, inter alia, in French Patents 1 222 944; 1 580 545; 2 265 782; 2 265 781; 1 564 110 and 2 439 798. A commercial product entering into this class is the resin 28-29-30 sold by the company NATIONAL STARCH.

D) the copolymers derived from C₄-C₈ monounsaturated carboxylic acids or anhydrides chosen from:

the copolymers comprising (i) one or more itaconic, fumaric or maleic acids or anhydrides and (ii) at least one monomer chosen from vinyl esters, vinyl ethers, vinyl halides, phenylvinyl derivatives, acrylic acid and its esters, the anhydride functions of these copolymers being optionally monoesterified or monoamidated. Such polymers are described in particular in U.S. Pat. Nos. 2,047,398; 2,723,248; 2,102,113 and Patent GB 839805. Marketed products are especially those sold under the names GANTREZ® AN or ES by the company ISP.

the copolymers comprising (i) one or more maleic, citraconic or itaconic anhydride units and (ii) one or more monomers chosen from allyl or methallyl esters optionally containing one or more acrylamide, methacrylamide or α-olefin groups, acrylic or methacrylic esters, acrylic or methacrylic acid or vinylpyrrolidone in their chain, the anhydride functions of these copolymers being optionally monoesterified or monoamidated.

These polymers are, for example, described in French Patents 2 350 384 and 2 357 241 by the applicant.

E) the polyacrylamides containing carboxylate groups.

The polymers comprising sulphonic groups are polymers containing vinylsulphonic, styrenesulphonic, naphthalenesulphonic or acrylamidoalkylsulphonic units.

These polymers may be especially chosen from:

the salts of polyvinylsulphonic acid having a molecular mass of between about 1000 and 100 000 as well as the copolymers with an unsaturated comonomer such as acrylic or methacrylic acids and their esters as well as acrylamide or its derivatives, vinyl ethers and vinylpyrrolidone;

the salts of polystyrenesulphonic acid such as the sodium salts sold for example under the name Flexan 130 by NATIONAL STARCH. These compounds are described in Patent FR 2 198 719.

the salts of polyacrylamidesulphonic acids, such as those mentioned in U.S. Pat. No. 4,128,631 and more particularly polyacrylamidoethylpropanesulphonic acid.

According to the invention, among the anionic fixing polymers cited above, those which are preferred are the acrylic acid copolymers such as the terpolymers acrylic acid/ethyl acrylate/N-tert-butylacrylamide sold under the name ULTRAHOLD® STRONG by the company BASF, the copolymers derived from crotonic acid such as the terpolymers vinyl acetate/vinyl tert-butylbenzoate/crotonic acid and the terpolymers crotonic acid/vinyl acetate/vinyl neododecanoate sold under the name Résine 28-29-30 by the company NATIONAL STARCH, the polymers derived from itaconic, fumaric and maleic acids or anhydrides with vinyl esters, vinyl ethers, vinyl halides, phenylvinyl derivatives, acrylic acid and its esters such as the monoesterified maleic anhydride/methylvinyl ether copolymers sold, for example, under the name GANTREZ® by the company ISP, the copolymers of methacrylic acid and of methyl methacrylate sold under the name EUDRAGIT® L by the company ROHM PHARMA, the copolymers of methacrylic acid and of ethyl acrylate sold under the name LUVIMER® MAEX or MAE by the company BASF and the copolymers vinyl acetate/crotonic acid and the copolymers vinyl acetate/crotonic acid grafted with polyethylene glycol sold under the name ARISTOFLEX® A by the company. BASF.

The anionic fixing polymers cited above which are most particularly preferred are those chosen from the monoesterified maleic anhydride/methylvinyl ether copolymers sold under the name GANTREZ® ES 425 by the company ISP, the terpolymers acrylic acid/ethyl acrylate/N-tert-butylacrylamide sold under the name ULTRAHOLD® STRONG by the company BASF, the copolymers of methacrylic acid and of methyl methacrylate sold under the name EUDRAGIT® L by the company ROHM PHARMA, the terpolymers vinyl acetate/vinyl tert-butylbenzoate/crotonic acid and the terpolymers crotonic acid/vinyl acetate/vinyl neododecanoate sold under the name Résine 28-29-30 by the company NATIONAL STARCH, the copolymers of methacrylic acid and ethyl acrylate sold under the name LUVIMER® MAEX or MAE by the company BASF, the terpolymers vinylpyrrolidone/acrylic acid/lauryl methacrylate sold under the name ACRYLIDONE® LM by the company ISP.

The fixing amphoteric polymers which can be used in accordance with the invention may be chosen from the polymers containing B and C units distributed statistically in the polymer chain where B denotes a unit which is derived from a monomer containing at least one basic nitrogen atom and C denotes a unit which is derived from an acidic monomer containing one or more carboxylic or sulphonic groups or alternatively B and C may denote groups which are derived from zwitterionic monomers of carboxybetaines or of sulphobetaines;

B and C may also denote a cationic polymer chain containing primary, secondary, tertiary or quaternary amine groups, in which at least one of the amine groups carries a carboxylic or sulphonic group linked via a hydrocarbon group or alternatively B and C form part of a chain of a polymer with an α,β-dicarboxylic ethylene unit in which one of the carboxylic groups has been caused to react with a polyamine containing one or more primary or secondary amine groups.

The amphoteric fixing polymers corresponding to the definition given above which are more particularly preferred are chosen from the following polymers:

(1) The polymers resulting from the copolymerization of a monomer derived from a vinyl compound carrying a carboxylic group such as more particularly acrylic acid, methacrylic acid, maleic acid, alpha-chloroacrylic acid, and of a basic monomer derived from a substituted vinyl compound containing at least one basic atom such as more particularly dialkylaminoalkyl methacrylate and acrylate, dialkylaminoalkylmethacrylamide and acrylamide. Such compounds are described in U.S. Pat. No. 3,836,537.

(2) The polymers containing units which are derived from:
a) at least one monomer chosen from acrylamides or methacrylamides substituted on the nitrogen atom by an alkyl group,
b) at least one acidic comonomer containing one or more reactive carboxylic groups, and
c) at least one basic comonomer such as esters with primary, secondary, tertiary and quaternary amine substituents of acrylic and methacrylic acids and the product of quaternization of dimethylaminoethyl methacrylate with dimethyl or diethyl sulphate.

The N-substituted acrylamides or methacrylamides more particularly preferred according to the invention are compounds whose alkyl groups contain from 2 to 12 carbon atoms and more particularly N-ethylacrylamide, N-tert-butylacrylamide, N-tert-octylacrylamide, N-octylacrylamide, N-decylacrylamide, N-dodecylacrylamide as well as the corresponding methacrylamides.

The acidic comonomers are chosen more particularly from acrylic, methacrylic, crotonic, itaconic, maleic and fumaric acids as well as the alkyl monoesters having 1 to 4 carbon atoms of maleic or fumaric anhydrides or acids.

The basic comonomers preferred are methacrylates of aminoethyl, butylaminoethyl, N,N′-dimethylaminoethyl, N-tert-butylaminoethyl.

Particularly used are the copolymers whose CTFA name (4th ed. 1991) is Octylacrylamide/acrylates/butylaminoethyl methacrylate copolymer such as the products sold under the name AMPHOMER® or LOVOCRYL® 47 by the company NATIONAL STARCH.

(3) The partially or completely acylated and crosslinked polyaminoamides derived from polyaminoamides of general formula: [symbol]
CO—R₁₀—CO-Z [symbol] (II)
in which R₁₀ represents a divalent group derived from a saturated dicarboxylic acid, a mono- or dicarboxylic aliphatic acid with ethylenic double bond, an ester of a lower alkanol having 1 to 6 carbon atoms of these acids or a group which is derived from the addition of any one of the said acids with a bis-primary or bis-secondary amine, and Z denotes a group which is derived from a bis-primary, mono- or bis-secondary polyalkylene polyamine and preferably represents:

a) in the proportions of 60 to 100 mol %, the group
—NH [symbol] (CH₂)ₓ—NH [symbol] ₚ (III)
where x=2 and p=2 or 3, or alternatively x=3 and p=2 this group being derived from the diethylenetriamine, triethylenetetraamine or dipropylenetriamine;

b) in the proportions of 0 to 40 mol %, the group (IIIbis) above, in which x=2 and p=1 and which is derived from ethylenediamine, or the group which is derived from piperazine:

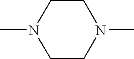

c) in the proportions of 0 to 20 mol %, the group —NH—(CH₂)₆—NH— which is derived from hexamethylenediamine,

these polyamino amides being crosslinked by adding a bifunctional crosslinking agent chosen from the epihalohydrins, diepoxides, dianhydrides, bis-unsaturated derivatives, by means of 0.025 to 0.35 mol of crosslinking agent per amine group of the polyamino amide and acylated by the action of acrylic acid, chloroacetic acid or of an alkanesultone or of their salts.

The saturated carboxylic acids are preferably chosen from the acids having 6 to 10 carbon atoms such as adipic, 2,2,4-trimethyladipic and 2,4,4-trimethyladipic acid, terephthalic acid, the acids with ethylene double bond such as for example acrylic, methacrylic and itaconic acids.

The alkanesultones used in the acylation are preferably propane or butanesultone, the salts of the acylating agents are preferably the sodium or potassium salts.

4) The polymers containing zwitterionic units of formula: 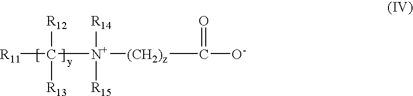
in which R₁₁ denotes a polymerizable unsaturated group such as an acrylate, methacrylate, acrylamide or methacrylamide group, y and z represent an integer from 1 to 3, R₁₂ and R₁₃ represent a hydrogen atom or a methyl, ethyl or propyl group, R₁₄ and R₁₅ represent a hydrogen atom or an alkyl group such that the sum of the carbon atoms in R₁₄ and R₁₅ does not exceed 10.

The polymers comprising such units may also comprise units derived from nonzwitterionic monomers such as dimethyl or diethylaminoethyl acrylate or methacrylate or alkyl acrylates or methacrylates, acrylamides or methacrylamides or vinyl acetate.

By way of example, there may be mentioned the copolymers of methyl methacrylate/methyl dimethylcarboxymethylammonioethyl methacrylate.

(5) The polymers derived from chitosan containing monomeric units corresponding to the following formulae: 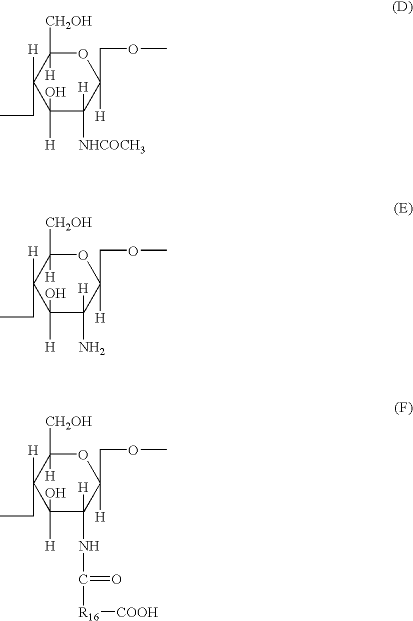
the D unit being present in proportions of between 0 and 30%, the E unit in proportions of between 5 and 50% and the F unit in proportions of between 30 and 90%, it being understood that in this F unit, R₁₆ represents a group of formula: 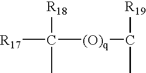
in which if q=0, R₁₇, R₁₈ and R₁₉, which are identical or different, each represent a hydrogen atom, a methyl, hydroxyl, acetoxy or amino residue, a monoalkylamine residue or a dialkylamine residue optionally interrupted by one or more nitrogen atoms and/or optionally substituted with one or more amine, hydroxyl, carboxyl, alkylthio or sulphonic groups, or an alkylthio residue whose alkyl group carries an amino residue, at least one of the R₁₇, R₁₈ and R₁₉ groups being in this case a hydrogen atom;

or if q=1, R₁₇, R₁₈ and R₁₉ each represent a hydrogen atom, as well as the salts formed by these compounds with bases or acids.

(6) The polymers corresponding to the general formula (V) are described for example in French Patent 1 400 366: 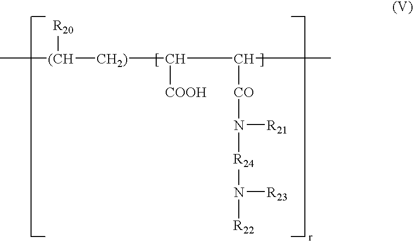
in which R₂₀ represents a hydrogen atom, a CH₃O, CH₃CH₂O or phenyl group, R₂₁ denotes a hydrogen atom or a lower alkyl group such as methyl or ethyl, R₂₂ denotes a hydrogen atom or a C₁-C₆ lower alkyl group such as methyl or ethyl, R₂₃ denotes a C₁-C₆ lower alkyl group such as methyl or ethyl or a group corresponding to the formula: —R₂₄—N(R₂₂)₂, R₂₄ representing a group —CH₂—CH₂—, —CH₂—CH₂—CH₂— or —CH₂—CH(CH₃)—, R₂₂ having the meanings mentioned above.

(7) The polymers derived from the N-carboxyalkylation of chitosan such as N-carboxymethyl chitosan or N-carboxybutyl chitosan.

(8) The amphoteric polymers of the -D-X-D-X— type chosen from:

a) the polymers obtained by the action of chloroacetic acid or sodium chloroacetate on the compounds containing at least one unit of formula:
-D-X-D-X-D- (VI)
where D denotes a group 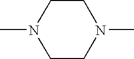
and X denotes the symbol E or E′, E or E′, which are identical or different, denote a bivalent group which is an alkylene group with a linear or branched chain containing up to 7 carbon atoms in the principal chain which is unsubstituted or substituted with hydroxyl groups and which may contain, in addition, oxygen, nitrogen or sulphur atoms, 1 to 3 aromatic and/or heterocyclic rings; the oxygen, nitrogen and sulphur atoms being present in the form of ether, thioether, sulphoxide, sulphone, sulphonium, alkylamine or alkenylamine groups, or hydroxyl, benzylamine, amine oxide, quaternary ammonium, amide, imide, alcohol, ester and/or urethane groups.

b) The polymers of formula:
-D-X-D-X- (VI′)
where D denotes a group 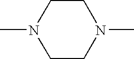
and X denotes the symbol E or E′ and, at least once, E′; E having the meaning indicated above and E′ is a bivalent group which is an alkylene group with a linear or branched chain having up to 7 carbon atoms in the principal chain, which is unsubstituted or substituted with one or more hydroxyl groups and containing one or more nitrogen atoms, the nitrogen atom being substituted with an alkyl chain optionally interrupted by an oxygen atom and necessarily containing one or more carboxyl functional groups or one or more hydroxyl functional groups and betainized by reaction with chloroacetic acid or sodium chloroacetate.

(9) The copolymers (C₁-C₅)alkyl vinyl ether/maleic anhydride partially modified by semiamidation with an N,N-dialkylaminoalkylamine such as N,N-dimethylaminopropylamine or by semiesterification with an N,N-dialkanolamine. These copolymers may also contain other vinyl comonomers such as vinylcaprolactam.

Among the amphoteric fixing polymers cited above which are most particularly preferred according to the invention, there may be mentioned those of the family (3) such as the copolymers whose CTFA name is Octylacrylamide/acrylates/butylaminoethyl methacrylate copolymer such as the products sold under the names AMPHOMER®, AMHOMER® LV 71 or LOVOCRYL® 47 by the company NATIONAL STARCH and those of the family (4) such as the methyl methacrylate/methyl dimethylcarboxymethylammonioethyl methacrylate copolymers.

The nonionic fixing polymers which can be used according to the present invention are chosen for example from:
polyalkyloxazolines;
homopolymers of vinyl acetate;
copolymers of vinyl acetate and acrylic ester;
copolymers of vinyl acetate and ethylene;
copolymers of vinyl acetate and maleic ester, for example dibutyl maleate;
the copolymers of acrylic esters such as for example the copolymers of alkyl acrylates and alkyl methacrylates such as the products offered by the company ROHM & HAAS under the names PRIMAL® AC-261 K and EUDRAGIT® NE 30 D, by the company BASF under the name 8845, by the company HOECHST under the name APPRETAN® N9212;
the copolymers of acrylonitrile and of a nonionic monomer chosen for example from butadiene and alkyl (meth)acrylates; there may be mentioned the products offered under the name CJ 0601 B by the company ROHM & HAAS;
the homopolymers of styrene;
the copolymers of styrene and alkyl (meth)acrylate such as the products MOWILITH® LDM 6911, MOWILITH® DM 611 and MOWILITH® LDM 6070 offered by the company HOECHST, the products RHODOPAS® SD 215 and RHODOPAS® DS 910 offered by the company RHONE POULENC;
the copolymers of styrene, alkyl methacrylate and alkyl acrylate;
the copolymers of styrene and butadiene;
the copolymers of styrene, butadiene and vinylpyridine;
the copolymers of alkyl acrylate and urethane;
the polyamides;
the homopolymers and copolymers of vinyllactam.

The alkyl groups of the nonionic polymers mentioned above preferably have from 1 to 6 carbon atoms.

According to the present invention, the nonionic fixing polymers with vinyllactam units may be those described in U.S. Pat. No. 3,770,683, U.S. Pat. No. 3,929,735, U.S. Pat. No. 4,521,504, U.S. Pat. No. 5,158,762, U.S. Pat. No. 5,506,315 and in patent applications WO 94/121148, WO 96/06592 and WO 96/10593. They may be provided in pulverulent form or in the form of a solution or a suspension.

The homopolymers or copolymers with vinyllactam units comprise units of formula (IX): 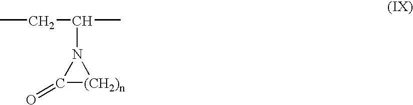
in which n is independently 3, 4 or 5.

The number-average molecular mass of the polymers with vinyllactam units is generally greater than about 5000, preferably between 10 000 and 1 000 000 approximately, more preferably between 10 000 and 100 000 approximately.

Among these fixing polymers, there may be mentioned polyvinylpyrrolidones such as those marketed under the name Luviskol® K30 by the company BASF; polyvinylcaprolactams such as those marketed under the name Luviskol® PLUS by the company BASF; poly(vinylpyrrolidone/vinyl acetate) copolymers such as those marketed under the name PVPVA® S630L by the company ISP, Luviskol® VA 73, VA 64, VA 55, VA 37 and VA 28 by the company BASF; and poly(vinylpyrrolidone/-vinyl acetate/vinyl propionate) terpolymers such as for example those marketed under the name Luviskol® VAP 343 by the company BASF.

The cationic film-forming polymers which can be used according to the present invention are preferably chosen from polymers containing primary, secondary, tertiary and/or quaternary amine groups forming part of the polymer chain or directly linked thereto, and having a molecular weight of between 500 and about 5 000 000 and preferably between 1000 and 3 000 000.

Among these polymers, there may be mentioned more particularly the following cationic polymers:

(1) the homopolymers or copolymers derived from acrylic or methacrylic esters or amides and comprising at least one of the units of the following formulae: 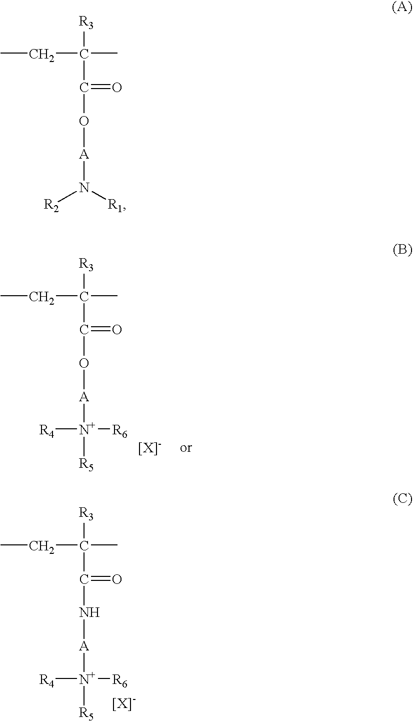
in which:
R₃ denotes a hydrogen atom or a CH₃ radical;
A is a linear or branched alkyl group of 1 to 6 carbon atoms, or a hydroxyalkyl group of 1 to 4 carbon atoms;
R₄, R₅, R₆, which are identical or different, represent an alkyl group having from 1 to 18 carbon atoms or a benzyl radical;
R₁ and R₂ represent hydrogen or an alkyl group having from 1 to 6 carbon atoms;
X denotes a methosulphate anion or a halide such as chloride or bromide.

The copolymers of the family (1) contain, in addition, one or more units derived from comonomers which may be chosen from the family of acrylamides, methacrylamides, diacetone acrylamides, acrylamides and methacrylamides substituted on the nitrogen with lower alkyls, acrylic or methacrylic acids or esters thereof, vinyllactams such as vinylpyrrolidone or vinylcaprolactam, vinyl esters.

Thus, among these copolymers of the family (1), there may be mentioned:

the copolymers of acrylamide and dimethylaminoethyl methacrylate quaternized with dimethyl sulphate or with a dimethyl halide such as that sold under the name HERCOFLOC by the company HERCULES,

the copolymers of acrylamide and methacryloyloxyethyltrimethylammonium chloride described, for example, in Patent Application EP-A-080976 and sold under the name BINA QUAT P 100 by the company CIBA GEIGY,

the copolymer of acrylamide and methacryloyloxyethyltrimethylammonium methosulphate sold under the name RETEN by the company HERCULES,

the vinylpyrrolidone/dialkylaminoalkyl acrylate or methacrylate copolymers, quaternized or otherwise, such as the products sold under the name “GAFQUAT” by the company ISP such as for example “GAFQUAT 734” or “GAFQUAT 755” or alternatively the products called “COPOLYMER 845, 958 and 937”. These polymers are described in detail in French Patents 2 077 143 and 2 393 573,

the dimethylaminoethyl methacrylate/vinylcaprolactam/vinylpyrrolidone terpolymers such as the product sold under the name GAFFIX VC 713 by the company ISP,

and the quaternized vinylpyrrolidone/dimethylaminopropyl methacrylamide copolymer such as the product sold under the name “GAFQUAT HS 100” by the company ISP.

(2) The quaternized polysaccharides described more particularly in U.S. Pat. Nos. 3,589,578 and 4,031,307 such as guar gums containing cationic trialkylammonium groups.

Such products are marketed in particular under the trade names JAGUAR C13S, JAGUAR C 15, JAGUAR C 17 by the company MEYHALL.

(3) The quaternary copolymers of vinylpyrrolidone and vinylimidazole such as the products marketed by BASF under the name Luviquat TFC;

(4) The chitosans or their salts;

the salts which can be used are in particular chitosan acetate, lactate, glutamate, gluconate or pyrrolidone carboxylate.

Among these compounds, there may be mentioned the chitosan having a degree of deacetylation of 90.5% by weight sold under the name KYTAN BRUT STANDARD by the company ABER TECHNOLOGIES, the chitosan pyrrolidonecarboxylate sold under the name KYTAMER PC by the company AMERCHOL.

(5) Cationic cellulose derivatives such as cellulose copolymers or cellulose derivatives grafted with a quaternary ammonium water-soluble monomer, and described especially in U.S. Pat. No. 4,131,576, such as hydroxyalkyl celluloses like hydroxymethyl, hydroxyethyl or hydroxypropyl celluloses grafted especially with a methacryloylethyltrimethylammonium, methacrylamidopropyltrimethylammonium or dimethyldiallylammonium salt.

The marketed products corresponding to this definition are more particularly the products sold under the name “CELQUAT L 200” and “CELQUAT H 100” by the company NATIONAL STARCH.

In the present invention, it is possible to use, as fixing polymer, hydrocarbon polymers with silicone grafts and silicones with hydrocarbon grafts. It is also possible to use polyurethanes. These various compounds may be nonionic, cationic, anionic or amphoteric.

A fixing polyurethane used in the present invention may advantageously additionally comprise at least one polysiloxane block or its basic repeating unit corresponds for example to general formula (VII):
—O—P—O—CO—NH—R—NH—CO— (VII)
in which:
P is a polysiloxane segment, and
R is a divalent group chosen from alkylene groups of the aromatic, C₁ to C₂₀ aliphatic, C₁ to C₂₀ cycloaliphatic type, these groups being substituted or unsubstituted.

Advantageously, the polysiloxane segment P corresponds to the general formula (VIII) below: 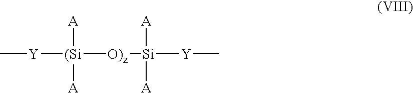
in which:
the groups A, which may be identical or different, are chosen, on the one hand, from the C₁ to C₂₀ monovalent hydrocarbon groups free or substantially free of ethylenic unsaturation and, on the other hand, aromatic groups,
Y represents a divalent hydrocarbon group, and
z represents an integer, chosen such that the average molecular mass of the polysiloxane segment is between 300 and 10 000.

In general, the divalent group Y is chosen from the alkylene groups of formula —(CH₂)ₐ— in which a represents an integer which may be between 1 and 10.

The groups A may be chosen from alkyl groups, in particular methyl, ethyl, propyl, isopropyl, butyl, pentyl, hexyl, octyl, decyl, dodecyl and octadecyl groups; cycloalkyl groups, in particular cyclohexyl group; aryl groups, especially phenyl and naphthyl groups; arylalkyl groups, especially benzyl and phenylethyl groups, and tolyl and xylyl groups.

By way of example of fixing polyurethane, there may be mentioned in particular the dimethylolpropionic acid/isophorone diisocyanate/neopentylglycol/polyesterdiols copolymer (also known by the name of polyurethane-1, INCI name) sold under the trade mark Luviset® PUR by the company BASF, the dimethylolpropionic acid/isophorone diisocyanate/neopentylglycol/polyesterdiols/silicone diamine copolymer (also known by the name of polyurethane-6, INCI name) sold under the trade mark Luviset® PUR A by the company BASF.

The hair compositions for hair styling according to the invention preferably comprises the fixing polymers in a quantity of between 0.1 and 20% by weight, preferably between 0.5% and 15% by weight relative to the total weight of the composition according to the invention.

The propellant preferably used in the present invention is dimethyl ether preferably in proportions of 20 to 60% of the total weight of the composition.

The cosmetically acceptable medium preferably comprises water and optionally a cosmetically acceptable solvent. This cosmetically acceptable solvent is chosen in particular from C₁-C₄ low alcohols such as ethanol, isopropanol, tert-butanol, n-butanol; polyols such as propylene glycol, and polyol ethers; acetone; and mixtures thereof, the particularly preferred solvent being ethanol.

The proportion of water may preferably be between 20 and 95% by weight relative to the total weight of the compositions. Advantageously, the medium is an aqueous-alcoholic mixture. The proportion of alcohol in the mixture is between 0 and 70% by weight, preferably between 0 and 55% by weight and still more preferably between 10 and 55% by weight relative to the total weight of the compositions.

The fixing hair composition according to the invention may additionally contain at least one adjuvant chosen from silicones in soluble, dispersed or microdispersed form, nonionic, anionic, cationic and amphoteric surfactants, ceramides and pseudoceramides, vitamins and provitamins including panthenol, vegetable, animal, mineral and synthetic oils, waxes other than ceramides and pseudoceramides, silicone or nonsilicone, water-soluble and fat-soluble sunscreens, coloured or noncoloured, inorganic and organic pigments, colorants, pearlescent and opacifying agents, sequestering agents, plasticizing agents, solubilizing agents, acidifying agents, alkalinizing agents, inorganic and organic thickening agents, antioxidants, hydroxy acids, penetrating agents, perfumes and preservatives.

Persons skilled in the art will be careful to choose the optional additives and their quantity such that they preferably do not impair the properties of the compositions of the present invention.

These additives are present in the composition according to the invention in a quantity ranging from 0 to 20% by weight relative to the total weight of the composition.

The compositions in accordance with the invention may be used as compositions for fixing and/or holding the hairs hair care compositions, shampoos, hair conditioning compositions, such as compositions intended to make the hair soft, or alternatively hair makeup compositions.

The present invention also relates to the use of the device for applying a lacquer to the hair by spraying its contents.

The present invention also relates to the use of the product sprayed by the aerosol device according to the invention as a hair lacquer.

The present invention also relates to a method of treatment, wherein the hair composition for hair styling contained in the aerosol device according to any one of the preceding claims is sprayed onto the hair which is wet or not.

The following examples illustrate the present invention and should not be considered in any manner as limiting the invention.

### Example 1

A composition in accordance with the invention formulated as follows:

| | |
|---|---|
| Luvitec VPI 55 K 61 | 4% |
| Water | 30% |
| Ethanol | 31% |
| DME | 35% |

### Example 2

A composition in accordance with the invention formulated as follows:

| | |
|---|---|
| Luvitec VPI 55 K 61 (AM) | 1% |
| Conventional polymer (Luviset ® PUR) (AM) | 4% |
| Water | 40% |
| Ethanol | 20% |
| DME | 35% |

AM: active material

The compositions of Examples 1 and 2, when packaged in an aerosol device and sprayed onto the hair, confer thereto very good holding associated with a very good cosmetic level.

The above written description of the invention provides a manner and process of making and using it such that any person skilled in this art is enabled to make and use the same, this enablement being provided in particular for the subject matter of the appended claims, which make up a part of the original description and including an aerosol device containing a hair composition for hair styling which comprises, in a cosmetically acceptable aqueous medium, at least one copolymer based on N-vinylpyrrolidone and N-vinylimidazole units having a molecular weight greater than 100 KDaltons, and dimethyl ether as propelling agent. Similarly described and enabled is a product containing two components, wherein the first component consists of a composition comprising, in a cosmetically acceptable aqueous medium, at least one copolymer based on N-vinylpyrrolidone and N-vinylimidazole units having a molecular weight greater than 100 KDaltons and in that the second component comprises a propelling agent consisting of dimethyl ether, the whole being packaged in an aerosol device, and a method of hair treatment, wherein the invention hair composition for hair styling contained in an aerosol device according is sprayed onto the hair which is wet or not.

As used above, the phrases “selected from the group consisting of,” “chosen from,” and the like include mixtures of the specified materials. The phrases “between X and Y” and “from X to Y” are inclusive of the endpoints.

All references, patents, applications, tests, standards, documents, publications, brochures, texts, articles, etc. mentioned herein are incorporated herein by reference. Where a numerical limit or range is stated, all values and subranges therewithin are specifically included as if explicitly written out.

The above description is presented to enable a person skilled in the art to make and use the invention, and is provided in the context of a particular application and its requirements. Various modifications to the preferred embodiments will be readily apparent to those skilled in the art, and the generic principles defined herein may be applied to other embodiments and applications without departing from the spirit and scope of the invention. Thus, this invention is not intended to be limited to the embodiments shown, but is to be accorded the widest scope consistent with the principles and features disclosed herein.

1. An aerosol device containing a hair composition, said composition comprising, in a cosmetically acceptable aqueous medium, at least one copolymer comprising N-vinylpyrrolidone and N-vinylimidazole units and having a molecular weight greater than 100 KDaltons, and dimethyl ether as propelling agent.

2. Aerosol device according to claim 1, wherein the copolymer has a molecular weight of between 100 and 5000 KDaltons.

3. Device according to claim 2, wherein the copolymer has a molecular weight of between 500 and 1500 KDaltons.

4. Device according to claim 1, wherein the copolymer consists of 10 to 90% by weight of N-vinylpyrrolidone units and of 10 to 90% by weight of N-vinylimidazole units.

5. Device according to claim 4, wherein the copolymer consists of 40 to 60% by weight of N-vinylpyrrolidone units and of 60 to 40% by weight of N-vinylimidazole units.

6. Device according to claim 1, wherein the composition comprises from 0.1 to 15% by weight of copolymer based on N-vinylpyrrolidone and N-vinylimidazole units relative to the total weight of the composition.

7. Device according to claim 1, wherein the composition further comprises a fixing polymer different from the copolymer based on N-vinylpyrrolidone and N-vinylimidazole units.

8. Device according to claim 7, wherein the fixing polymer is anionic, cationic, amphoteric or nonionic.

9. Device according to claim 7, wherein the additional fixing polymer is selected from the group consisting of the terpolymer crotonic acid/ethyl acrylate/N-tert-butylacrylamide, the terpolymer crotonic acid/vinyl acetate/vinyl tert-butylbenzoate, the terpolymer methacrylic acid/ethyl acrylate/tert-butyl acrylate, graft silicone polymers containing a polysiloxane backbone grafted with at least one hydrocarbon radical, polyurethanes, and mixtures thereof.

10. Device according to claim 7, wherein the composition comprises from 0.1 to 20% by weight of additional fixing polymer relative to the total weight of the composition.

11. Device according to claim 1, wherein the composition comprises from 20 to 60% of dimethyl ether of the total weight of the composition.

12. Device according to claim 1, wherein the cosmetically acceptable aqueous medium comprises water and a cosmetically acceptable solvent.

13. Device according to claim 12, wherein the solvent is selected from the group consisting of C₁-C₄ alcohols, polyols, polyol ethers, acetone and mixtures thereof.

14. Device according to claim 13, wherein the solvent is a C₁-C₄ alcohol.

15. Device according to claim 12, wherein the composition comprises from 20 to 95% of water by weight relative to the total weight of the composition.

16. Device according to claim 13, wherein the composition comprises from 0 to 70% by weight of C₁-C₄ alcohol relative to the total weight of the composition.

17. Device according to claim 1, wherein the composition further comprises an adjuvant selected from the group consisting of silicones in soluble, dispersed or microdispersed form, nonionic, anionic, cationic and amphoteric surfactants, ceramides and pseudoceramides, vitamins and provitamins including panthenol, vegetable, animal, mineral and synthetic oils, waxes other than ceramides and pseudoceramides, silicone or nonsilicone, water-soluble and fat-soluble sunscreens, coloured or noncoloured, inorganic and organic pigments, colorants, pearlescent and opacifying agents, sequestering agents, plasticizing agents, solubilizing agents, acidifying agents, alkalinizing agents, inorganic and organic thickening agents, antioxidants, hydroxy acids, penetrating agents, perfumes, preservatives, and mixtures thereof.

18. The device according to claim 1, wherein said composition is a hair lacquer.

19. A product containing two components, wherein the first component is a composition comprising, in a cosmetically acceptable aqueous medium, at least one copolymer based on N-vinylpyrrolidone and N-vinylimidazole units having a molecular weight greater than 100 KDaltons, and wherein the second component comprises a propelling agent comprising dimethyl ether, the product being packaged in an aerosol device.

20. A method of hair treatment, comprising spraying onto wet or dry hair the composition contained in the aerosol device according to claim 1 from said device.
